(19) United States
(12) Patent Application Publication
(10) Pub. No.: US 2017/0224852 A1
(43) Pub. Date: Aug. 10, 2017

(54) BENZAZEPIN-1-OL-DERIVED PET LIGANDS WITH HIGH IN VIVO NMDA SPECIFICITY

(71) Applicants: ETH ZURICH, Zurich (CH); Westfaelische Wilhelms - Universitaet, Muenster (DE); University of Zurich, Zurich (CH)

(72) Inventors: Simon M. Ametamey, Zurich (CH); Stefanie Kraemer, Zurich (CH); Ahmed Haider, Zug (CH); Linjing Mu, Lenzburg (CH); Bernhard Wuensch, Muenster (DE)

(21) Appl. No.: 15/427,952

(22) Filed: Feb. 8, 2017

Related U.S. Application Data
(60) Provisional application No. 62/292,365, filed on Feb. 8, 2016.

(51) Int. Cl. A61K 51/04

(52) U.S. Cl. CPC A61K 51/0468

(57) ABSTRACT

The present invention is directed to benzazepin-1-ol-derived compounds for use in the diagnosis of NMDA receptor-associated diseases or disorders by positron emission tomography (PET). The invention also relates to a method for the diagnosis of NMDA-receptor-associated diseases or disorders by administering to a patient in need of such diagnosis a radioactively labelled compound of the invention in an amount effective for PET imaging of NMDA receptors, recording at least one PET scan, and diagnosing an NMDA-receptor-associated disease or disorder from an abnormal NMDA receptor expression pattern on the PET scan. NMDA-receptor-associated diseases or disorders that can be diagnosed with the radioactively labelled benzazepin-1-ol-derived compounds include but are not limited to neurodegenerative diseases or disorders, Alzheimer's disease, depressive disorders, Parkinson's disease, traumatic brain injury, stroke, migraine, alcohol withdrawal and chronic and neuropathic pain.

### CROSS-REFERENCE TO RELATED APPLICATIONS

This application claims the benefit of, and priority to, U.S. Provisional Patent Application Ser. No. 62/292,365 filed Feb. 8, 2016, titled “Benzazepin-1-OL-Derived Pet Ligands With High In Vivo NMDA Specificity”, which is incorporated herein by reference in its entirety.

### FIELD OF THE INVENTION

The present invention is directed to benzazepin-1-ol-derived compounds for use in the diagnosis of NMDA (N-methyl-D-aspartate) receptor-associated diseases or disorders by positron emission tomography (PET). The invention also relates to a method for the diagnosis of NMDA receptor-associated diseases or disorders by administering to a patient in need of such diagnosis a radioactively labelled compound of the invention in an amount effective for PET imaging of NMDA receptors, recording at least one PET scan, and diagnosing an NMDA receptor-associated disease or disorder from an abnormal NMDA receptor expression pattern on the PET scan. NMDA receptor-associated diseases or disorders that can be diagnosed with the radioactively labelled benzazepin-1-ol-derived compounds include but are not limited to neurodegenerative diseases or disorders, Alzheimer's disease, depresssive disorders, Parkinson's disease, traumatic brain injury, stroke, migraine, alcohol withdrawal and chronic and neuropathic pain.

### BACKGROUND OF THE INVENTION

The functional complexity of the NMDA receptor family and the diversity of ligand binding sites discovered in recent years offer a wide variety of options for modulating neuronal activity. However, recent experience including a few disappointing clinical trials have shown that this complexity at the same time renders NMDA receptors challenging targets in drug development (Monaghan et al., Neurochem. Int. 2012, 61, 581-592; Curr Opin Pharmacol 2015, 20, 14-23). A functional NMDA receptor consists of four subunits, involving two or three of the seven homologous gene products GluN1, GluN2A-D, GluN3A and 3B. The subunit composition is highly adaptive and depends on the macro- and microscopic location of the receptor, the developmental age, neuronal function and activity (Paoletti et al., Nat. Rev. Neurosci. 2013, 14, 383-400). Due to the diverse and sometimes opposite functions of the individual receptor heterotetramers, subtype-selective compounds became of considerable interest in drug development.

However, despite great efforts in drug research towards GluN1/GluN2B-selective NTD (N-terminal domain) ligands, results from clinical trials were disappointing and did not hold the expectations from basic and preclinical research (Ikonomidou and Turski, Lancet Neurol. 2002, 1, 383-386). Comparing results from in vitro and in vivo experiments, the discrepancies between experimental Ki values (affinity to GluN1/GluN2B receptors) and concentrations or doses that were required to induce a particular pharmacodynamic or pharmacological response are striking in many cases. Several in vitro experiments indicated binding affinities and pharmacodynamic effects of GluN1/GluN2B-selective NTD modulators in the low nanomolar concentration range while other work reported significant binding and receptor-related effects only in the high nanomolar or low micromolar range (Schepmann et al., J. Pharm. Biomed. Anal. 2010, 53, 603-608). Several of these studies showed a high and a low affinity interaction with native (mixed tetraheteromeric) receptors, independent of the absolute values, in agreement with the reported high and low affinity binding to recombinant GluN1/GluN2B and GluN1/GluN2A, respectively. Considering the high brain uptake of eliprodil with brain/plasma ratios of about 20 (Garrigou-Cadenne et al., J Pharmacokinet Biopharm 1995, 23, 147-161) and assuming low nanomolar binding affinity (Tewes et al., Chem Med Chem 2010, 5, 687-695), effective doses of eliprodil in preclinical in vivo studies were magnitudes higher than what would be expected sufficient to occupy a high portion of the GluN1/GluN2B NTD binding sites (Toulmond et al., Brain Res. 1993, 620, 32-41). Furthermore, while regional expression levels of the individual subunits of the NMDA receptors are known (Laurie et al., Brain Res Mol Brain Res 1997, 51, 23-32), the in vivo regional binding pattern of the GluN1/GluN2B NTD-selective drugs remains elusive. The contradictive findings encouraged academic and industrial research teams to develop modulators with improved pharmacodynamic properties, in particular regarding affinity and selectivity (Strong et al., Expert Opin. Ther. Pat. 2014, 24, 1349-1366; Tewes et al., Chem Med Chem 2010, 5, 687-695). In addition to an improved selectivity pattern of the modulators, methods are required to optimize dosage schemes towards optimal receptor subtype occupancy at minimal binding to off-targets including alternative NMDA receptor subtypes.

Tewes et al. (Chem Med Chem 2010, 5, 687-695) describes the synthesis and biological evaluation of 3-benzazepin-1-ols as NR2B-selective NMDA receptor antagonists. In competition assays using tritium-labeled Ifenprodil as radioligand, affinity towards NR2B-containing NMDA receptors in membrane homogenates of cells stably expressing recombinant human NR1a/NR2B receptors (dexamethasone-induced, ketamine (NMDA antagonist) stabilized for avoiding cell death) was demonstrated for a number of 3-benzazepin-1-ols. For investigating the selectivity of these 3-benzazepin-1-ol-derived NR2B ligands, the compounds were tested against the phencyclidine (PCP) binding site of the NMDA receptor and both σ (sigma) receptor subtypes (σ₁ and σ₂) in receptor binding studies with radioligands. The benzazepines do not show significant interactions with the PCP binding site but high selectivity for the polyamine binding site of the NMDA receptor. The affinities towards the σ₂-receptor was also generally low. With regard to the σ₁-receptor, it was shown that structural changes in the 3-benzazepines can shift the receptor profile from an NR2B-selective ligand to a σ₁-selective ligand. Even though the 3-benzazepines of Tewes et al. were considered promising high affinity and NR2B-selective NMDA receptor antagonists with a potential for therapeutic use, clinical trials did not establish sufficient therapeutic benefit for medical use (Addy et al., J Clin Pharmacol, 49, 856-864, 2009).

Positron emission tomography (PET) is a nuclear medicine, functional imaging technique that produces a three-dimensional image of functional processes in the body. The system detects pairs of gamma rays emitted indirectly by a positron-emitting radionuclide (tracer), which is introduced into the body on a biologically active molecule. Three-dimensional images of tracer concentration within the body are then constructed by computer analysis. In modern PET-CT scanners, three dimensional imaging is often accomplished with the aid of a CT X-ray scan performed on the patient during the same session, in the same machine. PET is a valuable technique for research, pharmacodynamic preclinical studies of potential medicaments and it is used regularly for diagnosing certain diseases and disorders.

Because the radioactive tracer forms part of a biologically active and target-specific molecule, the so-called PET ligand, the PET technique images the target-specific distribution of the tracer in healthy and diseased live tissue. Depending on the target specificity of the PET ligand an abnormal biodistribution of targets can be indicative of diseases and disorders. For example, PET imaging is useful for diagnosing tumors and sites of metastases (oncology), for imaging neurodegenerative disease such as Alzheimer's disease, for localizing a seizure focus, for imaging psychiatric disorders such as schizophrenia, substance abuse, mood disorders (neuroimaging), for imaging artherosclerosis and vascular diseases (cardiology), and for imaging bacterial infections.

It is a common problem of PET ligands that there is slow or insufficient biodistribution or even no passage of the ligands through certain tissues, e.g. the blood brain barrier. Furthermore, PET ligand specificity for the aimed target is regularly compromised by unspecific binding of the ligand to non-targeted proteins such as serum albumin. All these shortcomings lead to low quality PET images which lack proper signal to noise ratio or display artifacts.

There are many PET ligands for neuroimaging, e.g. ¹¹C and ¹⁸F-labelled compounds such as Raclopride, Fallypride, Desmethoxyfallypride for dopamine D2/D3 receptors, McN 5652 and DASB for serotonin transporters, Mefway for serotonin 5HT1A receptors, Nifene for nicotinic acetylcholine receptors and a number of amyloid protein-specific PET ligands. However, at present there are no NMDA-specific PET ligands with an in vivo specificity that is sufficiently high, i.e. much higher than 30%, to correctly reflect the NMDA receptor biodistribution in patients with NMDA receptor-associated diseases or disorders.

In view of the above, it is the objective of the present invention to provide new PET ligands with high NMDA receptor affinity and high NMDA receptor selectivity that are suitable for use in the diagnosis of NMDA receptor-associated diseases or disorders by positron emission tomography (PET) with good biodistribution, high signal to noise ratio and little artifact generation.

In a first aspect the objective of the present invention is solved by the following compounds of formula (I):

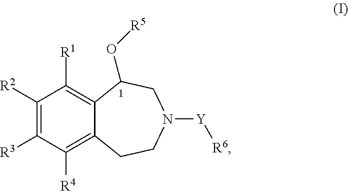

wherein
at least one atom of formula (I) is a radiolabeled atom, preferably a ¹¹C-, ¹⁸F-, ¹³N, or ¹⁵O atom, suitable for positron emission tomography detection (PET);
one of R¹, R², R³ and R⁴ is independently selected from the group consisting of hydrogen, fluorine, —(C₁-C₄)alkyl, fluorinated —(C₁-C₄)alkyl, preferably —CH₂F,—CD₂F, FCH₂CH₂—, FCH₂CH₂CH₂—,
O(C₁-C₄)alkyl, and fluorinated —O(C₁-C₄)alkyl, preferably —OCH₂F,—OCD₂F, FCH₂CH₂O—, and FCH₂CH₂CH₂O—,
and the other of R¹ to R⁴ are hydrogen or fluorine;
R⁵ is selected from the group consisting of hydrogen, —(C₁-C₆)alkyl, fluorinated —(C₁-C₆)alkyl, preferably —CH₂F,—CD₂F, FCH₂CH₂—, and FCH₂CH₂CH₂—;
Y is selected from the group consisting of
(C₁-C₆)alkyl, preferably C₅-alkyl or C₄-alkyl,
(C₁-C₆)alkoxyalkyl, (C₁-C₆)polyethyleneglycoyl, preferably —(CH₂)₂—O—(CH₂)₂—R⁶, —(CH₂)₃—O—R⁶, —(CH₂)₄—O—R⁶, and
(C₁-C₆)heteroalkyl, preferably —(CH₂)₃—X—R⁶ and —(CH₂)₄—X—R⁶, wherein X is sulfur or SO₂,

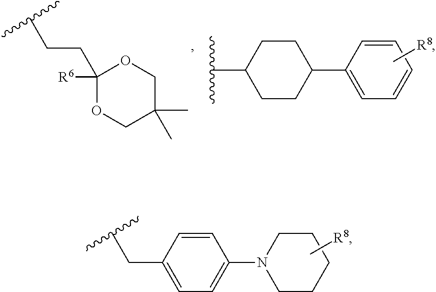

—(CH₂)₃—CO—R⁶, —(CH₂)₂—CO—N(CH₃)—CH₂—R⁶, and —CO—(CH₂)₃—R⁶, wherein R⁸ is one or more hydrogen or fluorine;
R⁶ is selected from the group consisting of
substituted or non-substituted (C₅-C₆)aryl, substituted or non-substituted (C₅-C₆)heteroaryl, preferably substituted or non-substituted phenyl or pyridyl,
more preferably

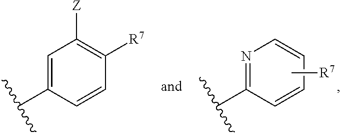

wherein Z is selected from the group consisting of hydrogen, fluorine and nitrile, wherein R⁷ is selected from the group consisting of hydrogen, fluorine, —(C₁-C₆)alkyl, fluorinated —(C₁-C₆)alkyl, preferably —CH₂F,—CD₂F, FCH₂CH₂—, FCH₂CH₂—, and FCH₂CH₂CH₂—;
and pharmaceutically acceptable salts or solvates thereof, for use in the diagnosis of NMDA receptor-associated diseases or disorders by positron emission tomography (PET).

In the context of the present invention it is understood that antecedent terms such as “alkyl” is to be interpreted as encompassing linear or branched, substituted or non-substituted alkyl residues. The scope of the term “linear or branched, substituted or non-substituted alkyl” encompasses linear or branched, substituted or non-substituted alkyl residues. For example, the term “(C₁₋₄)alkyl” indicates the group of compounds having 1 to 4 carbons that is linear or branched, substituted or non-substituted.

Alkoxyalkyl groups as used herein shall be understood to mean any linear or branched, substituted or non-substituted alkyl chain comprising an oxygen atom either as an ether motif, i.e. an oxygen bound by two carbons, as an oxygen bound to any other chemical atom than carbon, e.g. hydroxyl group, or an oxygen anion.

The term heteroatom as used herein shall be understood to mean atoms other than carbon and hydrogen such as and preferably O, N, S and P.

Heteroalkyl residues are carbon chains in which one or more carbon atoms can be optionally replaced by heteroatoms, preferably by 0, S or N. If N is not substituted it is NH. The heteroatoms may replace either terminal or internal carbon atoms within a linear or branched carbon chain. Such groups can be substituted as herein described by groups such as oxo to result in definitions such as but not limited to alkoxycarbonyl, acryl, amido and thioxo.

The term aryl as used herein shall be understood to mean an aromatic carbocycle or heteroaryl as defined herein. Each aryl or heteroaryl unless otherwise specified includes its partially or fully hydrogenated derivative. For example, quinolinyl may include decahydroquinolinyl and tetrahydroquinolinyl; naphthyl may include its hydrogenated derivatives such as tetrahydronaphthyl. Other partially or fully hydrogenated derivatives of the aryl and heteroaryl compounds described herein will be apparent to one of ordinary skill in the art. Naturally, the term encompasses aralkyl and alkylaryl, both of which are preferred embodiments for practicing the compounds of the present invention. For example, the term aryl encompasses phenyl, indanyl, indenyl, dihydronaphthyl, tetrahydronaphthyl, naphthyl and decahydronaphthyl.

The term heteroaryl shall be understood to mean an aromatic C₃-C₂₀, preferably 5-8 membered monoxyclic or preferably 8-12 membered bicyclic ring containing 1-4 heteroatoms such as N, O and S. Exemplary heteroaryls comprise aziridinyl, thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl, naphthyridinyl, indazolyl, triazolyl, pyrazolo[3,4-b]pyrimidinyl, purinyl, pyrrolo[2,3-b]pyridinyl, pyrazole[3,4-b]pyridinyl, tubercidinyl, oxazo[4,5-b]pyridinyl, and imidazo[4,5-b]pyridinyl. Terms which are analogues of the above cyclic moieties such as aryloxy or heteroaryl amine shall be understood to mean an aryl, heteroaryl, heterocycle as defined above attached to its respective group.

As used herein, the terms nitrogen and sulphur include any oxidized form of nitrogen and sulphur and the quaternized form of any basic nitrogen as long as the resulting compound is chemically stable. For example, for an —S—C₁₋₆ alkyl radical shall be understood to include —S(O)—C₁₋₆ alkyl and —S(O)₂—C₁₋₆alkyl.

The term polyethyleneglycol as used herein refers to a chain of substituted or non-substituted ethylene oxide monomers.

The compounds of the present invention are chemically stable and bind selectively to the NMDA receptor, in particular to the GluN2B subunit. Preferably, the compounds of the invention have an affinity to the NMDA receptor in the nanomolar range, preferably at least 100 nM, more preferably at least 10 nM, most preferably at least less than 5 nM. Assays for assessing NMDA receptor affinites are common general knowledge in the field and can be found, for example, in Tewes et al. (Chem Med Chem 2010, 5, 687-695) and in the Examples below.

The present PET ligands provide high-quality PET images in short scan times within 20-90 min and allow quantitative analysis of the ligand from the blood. Uptake of the present ligands into brain allows for non-invasive imaging of the density of NMDA receptors and enables to assess the degree of receptor occupancy by GluN1/GluN2B NTD modulators. The present PET ligands bind with high affinity in all major brain regions and biodistribution studies (see Examples below) demonstrated a heterogeneous accumulation of the present PET ligands in different regions of the rat brain which was reduced by up to 47% under blocking conditions. PET studies with the present ligands (see Examples below) after eliprodil administration at various doses revealed 50% receptor occupancy in GluN2B-rich brain regions at 1.1 to 1.7 μg/kg (3.1 to 5.0 nmol/kg). Receptors were occupied to 99% at 70 μg/kg which is by magnitudes (4 to 500-fold) lower than typical doses used in preclinical animal experiments. In the GluN2B-poor but GluN2D-rich regions midbrain and brain stem, receptors were occupied to 50% at 6.2 and 7.7 nmol/kg, respectively. No radioactive metabolites could be detected in the blood, urine and brain homogenate of rats, indicating metabolic stability of the present PET ligands. In conclusion, the present ligands allow for the first time to non-invasively image the density of GluN2B(C,D)-containing NMDA receptors in the mammalian brain and to assess the degree of receptor occupancy by GluN1/GluN2B NTD modulators.

In a preferred embodiment, the PET ligand for use in the present invention is one, wherein the at least one radiolabeled atom is a ¹¹C-atom or ¹⁸F-atom.

The radiolabeled atom can be introduced by radiosynthetic means known in the art (see Examples 2 and 3). Typically, the radiosynthesis of the present PET ligands can be accomplished within about 1 to 2 hours after end of bombardment and yields in about 2 to 11.3 GBq at the end of the synthesis.

In a more preferred embodiment the compound for use in the invention is one, wherein at least one of R¹, R³ or R⁵ comprise a ¹¹C-atom. Preferably, one of R¹, R³ is —O[¹¹C]H₃ or R⁵ is —[¹¹C]H₃.

In a further preferred embodiment one of R¹, R², R³ and R⁴ is independently selected from the group consisting of —CH₃, —CH₂F,—CD₂F, FCH₂CH₂—, FCH₂CH₂CH₂—, —OCH₃, —OCH₂F, —OCD₂F, FCH₂CH₂O— and FCH₂CH₂CH₂O—, and the other of R¹ to R⁴ are hydrogen or fluorine.

In a further preferred embodiment, R¹ is selected from the group consisting of —CH₃, —CH₂F,—CD₂F, FCH₂CH₂—, FCH₂CH₂CH₂—, —OCH₃, —OCH₂F, —OCD₂F, FCH₂CH₂O— and FCH₂CH₂CH₂O and R², R³ and R⁴ are hydrogen or fluorine

In a further preferred embodiment, R³ is selected from the group consisting of —CH₃, —CH₂F,—CD₂F, FCH₂CH₂—, FCH₂CH₂CH₂—, —OCH₃, —OCH₂F, —OCD₂F, FCH₂CH₂O— and FCH₂CH₂CH₂O and Fe, R² and R⁴ are hydrogen or fluorine.

In a further preferred embodiment, R⁵ is selected from the group consisting of hydrogen, —CH₂F,—CD₂F, FCH₂CH₂—, and FCH₂CH₂CH₂—.

In a further preferred embodiment, Y is selected from the group consisting of —(CH₂)ᵢ—R⁶ and —(CH₂)ₑ—O—(CH₂)ₑ—R⁶, wherein i is an integer from 2 to 6, and e is an integer from 1 to 3.

In a further preferred embodiment, R⁶ is selected from the group consisting of phenyl,

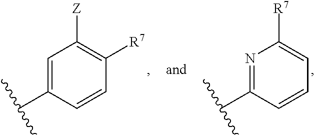

wherein Z is hydrogen or nitrile and wherein R⁷ is selected from the group consisting of fluorine, —CH₂F,—CD₂F, FCH₂CH₂—, FCH₂CH₂—, and FCH₂CH₂CH₂—.

In a more preferred embodiment the compound for use in the invention is one wherein the compound is R-configured at carbon 1.

In a further preferred embodiment the compound for use in the invention is one wherein the compound is 5-configured at carbon 1.

In a more preferred embodiment the compound for use in the invention is one wherein
R¹ is selected from the group consisting of —OCH₃, —OCH₂F,—OCD₂F, FCH₂CH₂O— and FCH₂CH₂CH₂O and R², R³ and R⁴ are hydrogen,
R⁵ is hydrogen,
Y is —(CH₂)₄—R⁶, and
R⁶ is substituted or un-substituted phenyl.

In a further preferred embodiment the compound for use in the invention is one wherein
R³ is selected from the group consisting of —OCH₃, —OCH₂F,—OCD₂F, FCH₂CH₂O— and FCH₂CH₂CH₂O and R¹, R² and R⁴ are hydrogen or fluorine,
R⁵ is selected from the group consisting of hydrogen, —CH₂F,—CD₂F, FCH₂CH₂—, and FCH₂CH₂CH₂—,
Y is —(CH₂)₄—R⁶ or —(CH₂)₂—O—(CH₂)₂—R⁶, and
R⁶ is selected from the group consisting of phenyl,

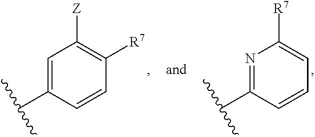

wherein Z is hydrogen or nitrile and wherein R⁷ is selected from the group consisting of fluorine, —CH₂F,—CD₂F, FCH₂CH₂—, FCH₂CH₂—, and FCH₂CH₂CH₂—.

In a further preferred embodiment the compound for use in the invention is one wherein
R³ is selected from the group consisting of fluorine, —CH₃, —CH₂F,—CD₂F, FCH₂CH₂— and FCH₂CH₂CH₂— and R¹, R² and R⁴ are hydrogen,
R⁵ is hydrogen,
Y is —(CH₂)₄—R⁶, and
R⁶ is substituted or un-substituted phenyl.

In a further preferred embodiment the compound for use in the invention is selected from the group consisting of

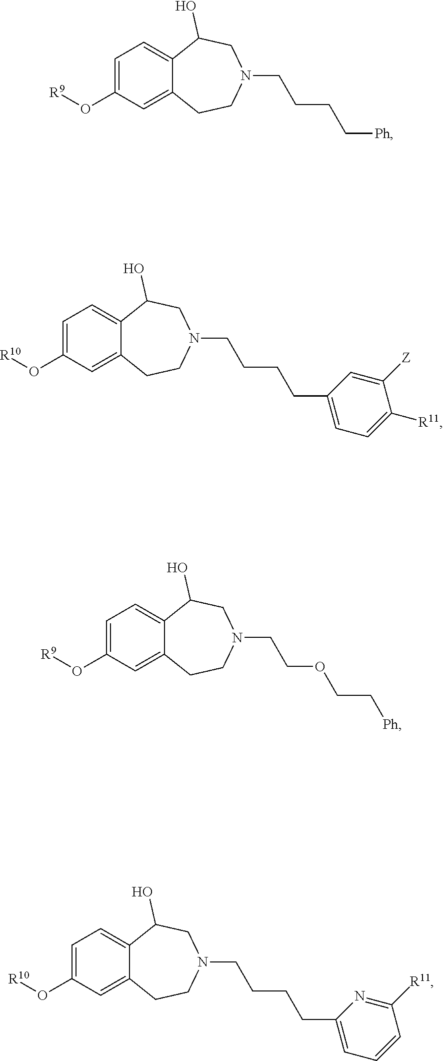

wherein
R⁹ is selected from the group consisting of CH₃, —CH₂F,—CD₂F, FCH₂CH₂—, FCH₂CH₂—, and FCH₂CH₂CH₂—;
R¹⁰ is selected from the group consisting of hydrogen, CH₃, —CH₂F,—CD₂F, FCH₂CH₂—, FCH₂CH₂—, and FCH₂CH₂CH₂—;
R¹¹ is selected from the group consisting of fluorine, —CH₂F,—CD₂F, FCH₂CH₂—, FCH₂CH₂—, and FCH₂CH₂CH₂—; and
Z is selected from the group consisting of hydrogen and nitrile.

The present invention includes pharmaceutically acceptable salts or solvates of the compounds of formula I. A “pharmaceutically acceptable salt or solvate” refers to any pharmaceutically acceptable salt or solvate which, upon administration to a patient, is capable of providing (directly or indirectly) a compound of the invention, or a pharmacologically active metabolite or pharmacologically active residue thereof. A pharmacologically active metabolite shall be understood to mean any compound of the invention capable of being metabolized enzymatically or chemically.

Pharmaceutically acceptable salts include those derived from pharmaceutically acceptable inorganic and organic acids and bases. Examples of suitable acids include hydrochloric, hydrobromic, sulphuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids. Other acids, such as oxalic acid, while not themselves pharmaceutically acceptable, may be employed in the preparation of salts useful as intermediates in obtaining the compounds and their pharmaceutically acceptable acid addition salts. Salts derived from appropriate bases include alkali metal (e.g., sodium), alkaline earth metal (e.g. magnesium), ammonium and N—(C₁-C₄alkyl)₄⁺ salts.

In addition, the scope of the invention also encompasses prodrugs of compounds of the formula I. Prodrugs include those compounds that, upon simple chemical transformation within a body of a patient, are modified to produce compounds of the invention. Simple chemical transformations include hydrolysis, oxidation and reduction. Specifically, when a prodrug is administered to a patient, the prodrug may be transformed into a compound disclosed hereinabove, thereby imparting the desired pharmacological effect.

Without wishing to be bound by theory, it is believed that the residues R5 and R6 in the compounds of formula I has a positive impact on NR2B-selective NMDA receptor binding, in particular NMDA receptor selectivity and substantially improves biodistribution of the PET ligands in the mammalian body and in particular in the brain. The improved selectivity together with the improved biodistribution makes the compounds of the present invention excellent candidates for diagnostic applications relating to the ECS in mammals.

In example 7 below experimental evidence demonstrates that NR2B ligands for use in the present invention are also suitable for indirectly imaging sigma (σ) receptor activity in mammalian tissue, in particular signal receptor activity. It is known that the signal receptor interacts with NMDA NR1A and modulates NMDA receptor function (Balasuriya D, J Neurosci. 2013, 33(46), 18219-24; Ishikawa et al., Biol Psychiatry 2007: 62:878-883). For this reason, the present invention also relates to the above-cited compounds of formula (I) and pharmaceutically acceptable salts or solvates thereof, for use in the identification of sigma receptors, preferably signal receptors, in particular in mammalian tissue, preferably in human, more preferably in live human tissue, most preferably in live human brain. Furthermore, the present invention is also directed to compounds of formula (I) and pharmaceutically acceptable salts or solvates thereof for use in the diagnosis of sigma receptor-associated diseases or disorders by positron emission tomography (PET).

In a further aspect, the present invention is directed to a method for the diagnosis of NMDA-receptor-associated diseases or disorders comprising the following steps:
(a) administering to a patient in need of such diagnosis a compound according to claim 1 in an amount effective for PET imaging of NMDA receptors,
(b) recording at least one PET scan,
(c) diagnosing an NMDA-receptor-associated disease or disorder from an abnormal NMDA receptor expression pattern on the PET scan.

In a preferred embodiment, the NMDA-receptor-associated disease or disorder is selected from the group consisting of neurodegenerative diseases or disorders, Alzheimer's disease, depressive disorders, Parkinson's disease, traumatic brain injury, stroke, migraine, alcohol withdrawal and chronic and neuropathic pain.

PET imaging of NMDA receptors in the human body, in particular the living human brain is a modern but already standard procedure in medical science and diagnosis. The average skilled person can routinely select an effective dosage, an effective formulation, the route and site of administration as well as all further parameters that are necessary to provide a meaningful PET scan of the respective positron-emitting tracer compound in mammalian tissue. For an overview on PET imaging in general and PET imaging of NMDA-associated diseases and disorders in particular, reference is made to the articles of Sobrio et al., Mini-reviews in Medicinal Chemistry, 10, 870-886, 2010; Asselin et al., NeuroImage, 22, T131, 2004; Bressan et al., Biol Psychiatry, 58, 41-46, 2005; and Hartwig et al., Clin Pharmacol. Ther, 58, 165-178, 1995.

For diagnostic use the compounds of the invention may be administered in any conventional dosage form in any conventional manner. Routes of administration include oral, intravenous, intramuscular and subcutaneous injections. The preferred modes of administration is intravenous.

The compounds may be administered alone or in combination with pharmaceutically acceptable excipients, e.g. excipients that enhance stability of the compounds, facilitate administration of pharmaceutical or diagnostic compositions containing them, provide increased dissolution or dispersion, diluents, buffers, viscosity-modifying agents, and the like, including other active ingredients. Advantageously such combination compositions utilize lower dosages of the conventional diagnostics, thus avoiding possible toxicity and adverse side effects incurred when those agents are used as monosubstances. The above-described compounds may be physically combined with conventional diagnostics or other excipients into a single pharmaceutical composition. Reference in this regard may be made to Cappola et al.: U.S. patent application Ser. No. 09/902,822, PCT/US 01/21860 and U.S. provisional application No. 60/313,527, each incorporated by reference herein in their entirety. Advantageously, the compounds of the invention may be administered alone or in combination with other biologically active compounds in a single or a multiple dosage form. The optimum percentage (w/w) of a compound of the invention in a dosage for PET Scanning may vary and is within the purview of those skilled in the art. Alternatively, the compounds for PET Scanning may be administered in several dosages.

As mentioned above, dosage forms of the compounds described herein include pharmaceutically acceptable excipients known to those of ordinary skill in the art. Methods for preparing such dosage forms are known (see, for example, H. C. Ansel and N. G. Popovish, Pharmaceutical Dosage Forms and Drug Delivery Systems, 5^{th} ed. Lea and Febiger (1990)). Dosage levels and requirements are well-recognized in the art and may be selected by those of ordinary skill in the art from available methods and techniques suitable for a particular patient. In some embodiments, dosage levels range from about 1-100 mg/dose for a 70 kg patient. Although one dose per PET scan may be sufficient, up to 2 doses per PET scan may be given. For intravenous doses, up to 2000 mg/PET scan may be required. Reference in this regard may also be made to U.S. provisional application No. 60/339,249. As the skilled artisan will appreciate, lower or higher doses may be required depending on particular factors. For instance, specific doses and diagnostic procedures will depend on factors such as the patient's general health profile, the severity and course of the patient's disorder or disposition thereto, and the judgment of the diagnosing physician.

In the following, the invention will be illustrated by way of specific examples, none of which are to be interpreted as limiting the scope of the claims as appended.

### BRIEF DESCRIPTION OF THE DRAWINGS

FIGS. 1A and 1B. FIG. 1A shows [¹¹C]NB1 binding to rat brain slices by in vitro autoradiography. Baseline and blocking conditions as indicated. The two slices were incubated and exposed simultaneously. FIG. 1B shows the analytical HPLC-profile of the enantiomeric separation of racemic NB1.

FIGS. 2A, 2B and 2C. FIG. 2A shows PET scans of rat brain with [¹¹C]WMS1405, averaged from 0 to 60 min p.i. FIG. 2B shows time-activity curves (TAC) of whole brain of the scans shown in FIG. 2A. FIG. 2C shows racemate baseline and blockade as in FIGS. 2A and 2B, and TAC of a displacement experiment with 1 mg/kg eliprodil 20 min after scan start/tracer injection.

FIGS. 3A and 3B show PET scans of rat brain with [¹⁸F]WMS1410. FIG. 3A. A(−) stereoisomer, FIG. 3B. B(+) stereoisomer. Baseline blockade (1 mg/kg eliprodil co-injection) and displacement (1 mg/kg eliprodil 20 min after scan start/tracer injection) scans.

FIGS. 4A and 4B show [¹¹C]WMS1405 indirectly images modulation of NMDA receptors by the signal receptor. FIG. 4A. In vitro displacement of [³H](+)pentazocine, a signal receptor ligand with Kd 1.6 nM (Lever et al., Synapse 2006, 59, 350-358.), by WMS1405, from its binding to rat brain membranes. One out of three independent experiments. Triplicate incubations with standard deviations. Solid line, fit IC50 function. Upper broken line, binding of 2.9 nM [³H](+)pentazocine alone. Lower broken line, blocking with 0.1 nM haloperidol. Ki of WMS1405 for signal receptor was 1-10 μM, indicating no significant binding to signal receptor. FIG. 4B In vivo, pentazocine and haloperidol reduced [¹¹C]WMS1405 binding to similar levels as under blocking conditions with eliprodil, despite the absence of competition at the signal receptor between WMS1405 and pentazocine in vitro. Baseline and blockade (circles) as in FIGS. 2B and 2C. Squares, co-injection of 2.5 mg/kg (+)-pentazocine. Haloperidol (0.13 mg/kg) co-injection with the tracer had a similar effect as (+)-pentazocine (data not shown).

### EXAMPLES

### Example 1—Chemistry

The general methodology for the synthesis of benzazepin-1-ols is known in the art, e.g. from Tewes et al., Chem Med Chem 2010, 5, 687-695. A representative synthetic path as used for producing the present PET ligands is shown in scheme 1 below. The synthetic route of scheme 1 can be adapted by commonly known methods to deliver derivatives of benzazepin-1-ols and substantially all of the PET ligands of the present invention. The skilled person will routinely adapt the synthetic route to be suitable for the synthesis of any PET ligand of the present invention.

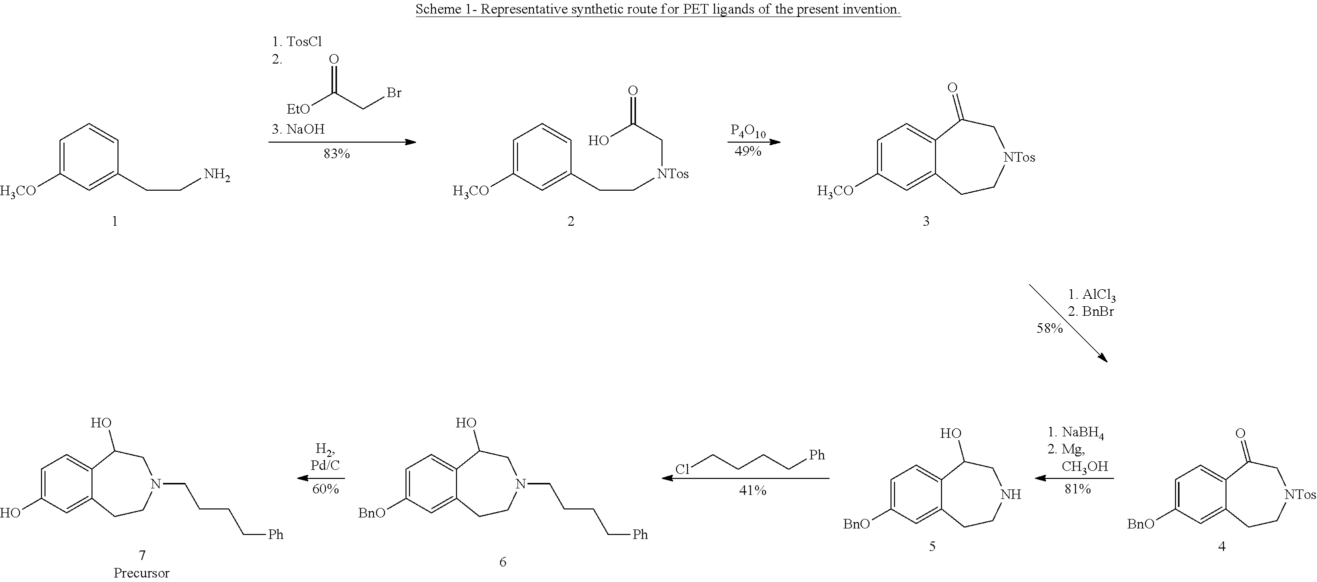

### Example 2—C-11 Radiolabeling

No-carrier-added (n.c.a.) [¹¹C]CO₂ was produced via the ¹⁴N(p,α)¹¹C nuclear reaction by bombardment of nitrogen gas fortified with 0.5% oxygen using a Cyclone 18/9 cyclotron (18-MeV, IBA, Louvain-la-neuve, Belgium). [¹¹C]Iodomethane ([¹¹C]CH₃I) was generated in a two-step reaction sequence involving the catalytic reduction of [¹¹C]CO₂ to [¹¹C]methane over a supported nickel catalyst and subsequent gas phase iodination [Larsen et al, Appl Radiat Isot, 48, 153-157, 1997]. [¹¹C]CH₃I was bubbled into the reaction vial containing precursor NB1 (1 mg) and cesium carbonate (5 mg) in DMF (0.5 mL). After the transfer was complete, the closed reaction vial was heated at 90° C. for 3 min. The reaction mixture was diluted with water (1.5 mL), and the crude product was purified using semi-preparative HPLC column (reversed-phase Sunfire C18 column Waters, Ireland, 5 μm, 10×150 mm, product peak at 12.8 min). The collected product was diluted with water (10 mL), trapped on a C18 cartridge (Waters, Ireland), preconditioned with 5 mL EtOH and 10 mL water), washed with water (5 mL) to remove traces of HPLC eluent. The product was eluted with 0.8 ml ethanol into a sterile pyrogen-free penicillin vial which contains 9.2 mL water for injection. For quality control, an aliquot of the formulated solution was injected into an analytical HPLC system. The identity of the ¹¹C-labeled product was confirmed by comparison with the retention time of its nonradioactive reference compound NB1 (5.9 min). The specific radioactivity was determined by matching the area under UV absorbance peak at 230 nm, which co-eluted with the radiolabeled product, to a standard calibration curve calculated using known concentrations of the non-radioactive reference compound NB1. ¹¹C-radiolabeling was achieved by reacting the phenolic salt of NB1 with [¹¹C]CH₃I in DMF. Typically, specific activities ranged between 290±90 GBq/μmol with a total activity of 7.4±1.9 GBq (ca. 60-72% radiochemical yield, decay corrected) at the end of synthesis (n=17) and a total synthesis time from end of bombardment was 35-40 min. The final product was of >99% radiochemical purity as confirmed by HPLC analysis. The identity of the tracer was confirmed by co-injection with non-radioactive Me-NB1.

The radiolabeling procedure was carried out identically for both enantiomers of the precursor NB1 and the obtained results for specific activity, total activity, synthesis time and radiochemical purity were in the same range when compared to the racemic mixture.

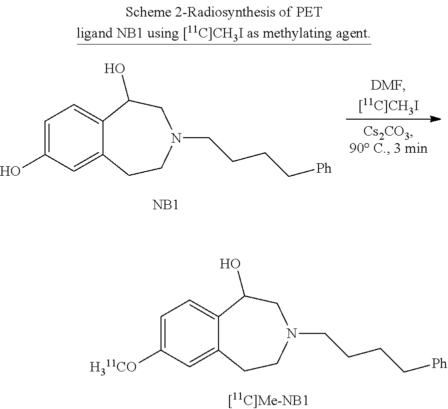

### Example 3—F-18 Radiolabeling

[¹⁸F]fluoride was produced by bombardment of enriched ¹⁸O-water using a Cyclone 18/9 cyclotron (18-MeV; IBA, Belgium). The aqueous ¹⁸F-fluoride was delivered from cyclotron to the hot cell and trapped on an anion exchange cartridge (Waters, Ireland, SepPak Accell QMA cartridge carbonate). After elution with a tetrabutylammonium hydroxide solution (0.18 M in MeOH, 1 mL) into a reaction vessel the solvents were evaporated at 90° C. under reduced pressure with a gentle inflow of nitrogen gas. Azeotropic drying was carried out three times using 1 mL of acetonitrile each.

A solution of 5 mg ethylene ditosylate in 0.5 mL acetonitrile was added and the reaction mixture was stirred at 100° C. for 7 min. After dilution with water (2.5 mL) the crude product was purified by semi-preparative HPLC column (Sunfire, Waters, Ireland, C18 column, 5 μm, 10×150 mm). [¹⁸F]fluoroethyl tosylate was diluted with 40 mL of water and trapped on a C18 light cartridge (Waters, Ireland, preconditioned with 5 mL EtOH and 10 mL water). The cartridge was washed with water (2 mL) and the [¹⁸F]fluoroethyl tosylate was eluted with 0.5 mL of N,N-dimethylformamide into a reaction vessel previously loaded with 1 mg of precursor 3-(4-phenylbutyl)-2,3,4,5-tetrahydro-1H-3-benzazepine-1,7-diol (NB1, which was synthesized according to the published synthetic route described by Tewes et. al, Chem Med Chem. 2010, 5, 687-95) and 5 mg of cesium carbonate in 0.2 mL of N,N-dimethylformamide. The reaction mixture was stirred for 120° C. for 10 min, diluted with water (2.3 mL) and purified by using the same semi-preparative HPLC column (product peak at 11.3 min). The collected product fraction of [¹⁸F]fluoroethyl-NB1 was diluted with water (15 mL) and trapped on a C18 light cartridge (Waters, Ireland, preconditioned with 5 mL EtOH and 10 mL water). After washing the cartridge with water (5 mL) the product was eluted with ethanol (0.5 mL) through a sterile filter (0.2 μm). The volume of ethanol was decreased to 0.1 mL under reduced pressure and the resulting solution diluted with 1.9 mL of water to give a final ethanol concentration of 5%. An aliquot of the final formulation was analyzed by using a reversed-phase column (Atlantis T3, Waters, Ireland, C18, particle size 3 μm, 4.6×150 mm) and the identity of the tracer was confirmed by comparison with the retention time of the non-radioactive reference compound (6.3 min). The specific activity was calculated by comparison of the UV (absorbance at 230 nm) peak area under the curve with a calibration curve of the reference compound. The total activity was 684±53 MBq (enantiomer A) and 621±364 MBq (enantiomer B) at the end of synthesis (n=2). The decay corrected radiochemical yield was 1.5±0.4% (enantiomer A) and 0.9±0.2% (enantiomer B) with a total synthesis time of 120-145 min. Radiochemical purity was >99% in all cases as confirmed by HPLC analysis and the identity of the tracer was confirmed by comparison with the retention time of the cold reference.

The radiolabeling procedure was carried out identically for both enantiomers of the precursor NB1.

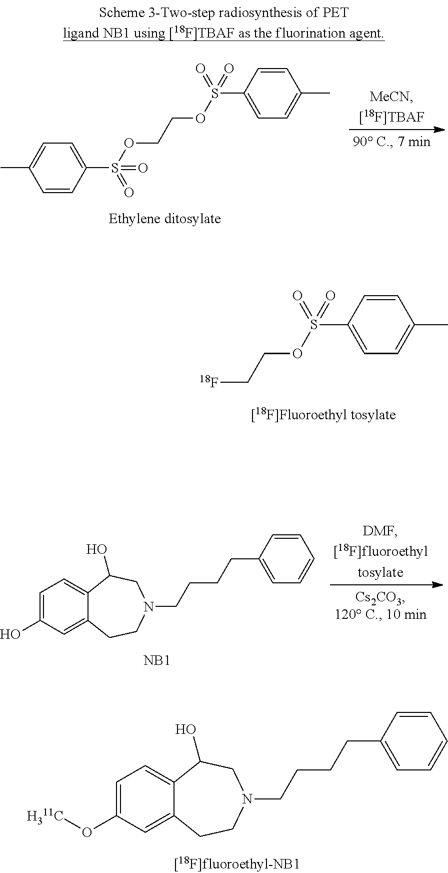

### Example 4—[¹¹C]NB1 Accumulates in Brain and is Displaced by GluN1/GluN2B NTD Modulators In Vitro

For in vitro autoradiography, horizontal rat brain slices (20 μm) of a male Wistar rat (492 g) were incubated for 30 min at room temperature with 5.4 nM [¹¹C]NB1 alone or in combination with 10 μM either eliprodil or AAM077 in HEPES buffer (30 mM HEPES, 110 mM NaCl, 5 mM KCl, 2.5 mM CaCl₂, 1.2 mM MgCl₂, pH 7.4) containing 0.1% bovine serum albumin (HEPES/BSA). After incubation, the slices were washed with HEPES/BSA for 8 min and twice with HEPES buffer for 3 min followed by two 5-second rinses with water. The dried slices were exposed for 20 min to a phosphorimager plate, read in a phosphorimager BAS5000 (Fuji, Tokyo, Japan) and analyzed with the software AIDA v. 4.5 (Raytest Isotopenmessgerate GmbH, Germany).

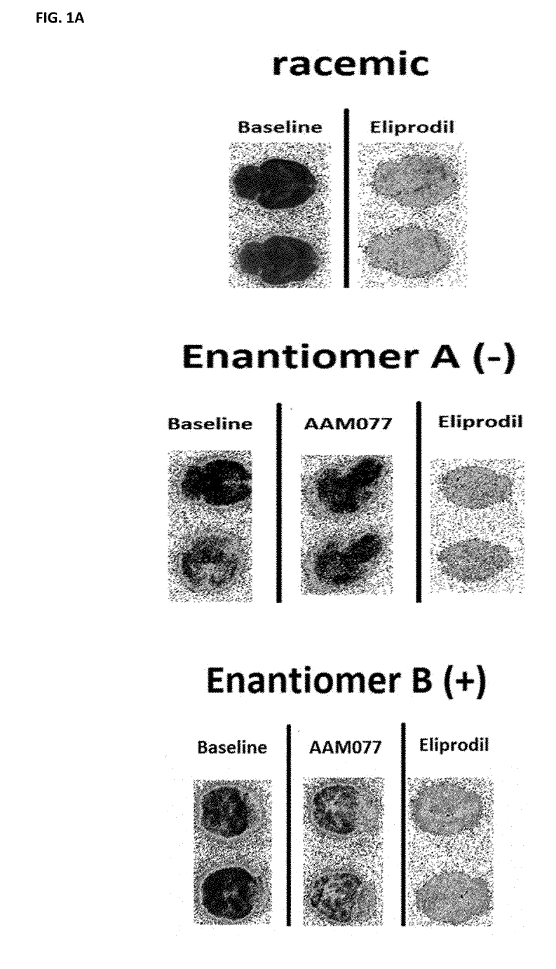

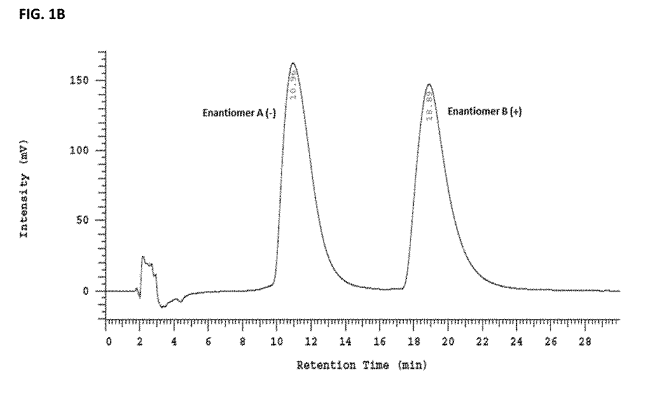

In vitro autoradiography with rat brain slices revealed high binding in most brain regions which was blocked by Glu2B specific ligand eliprodil but not AAM077 (Glu2A specific ligand, Novartis Pharmaceuticals, Switzerland). Typical autoradiographs are shown in FIG. 1A.

### Example 5—PET Scans of Rat Brain with [¹¹C]WMS1405, Averaged from 0 to 60 Min p.i

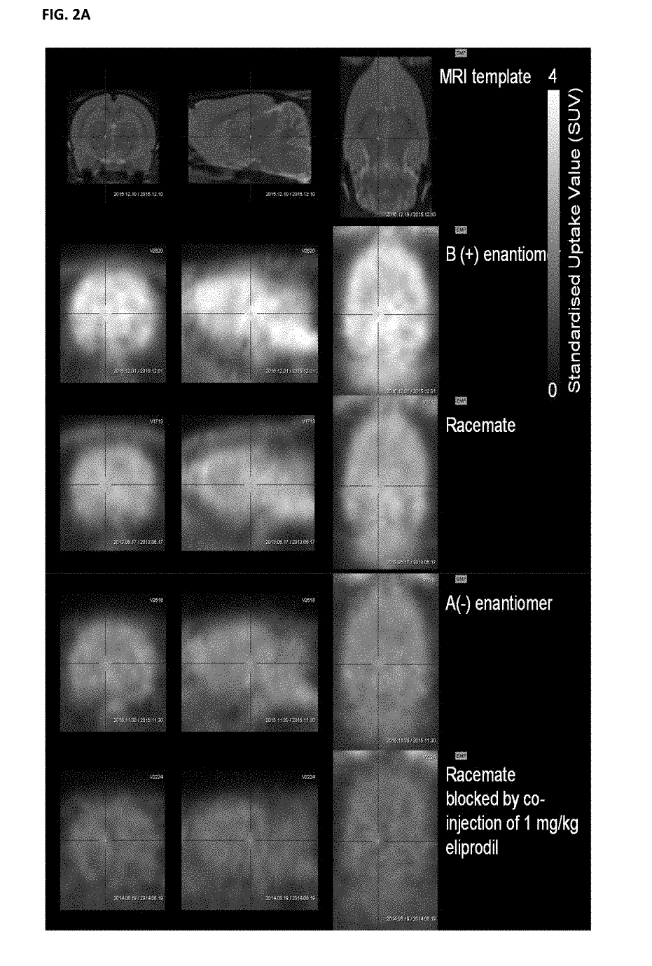

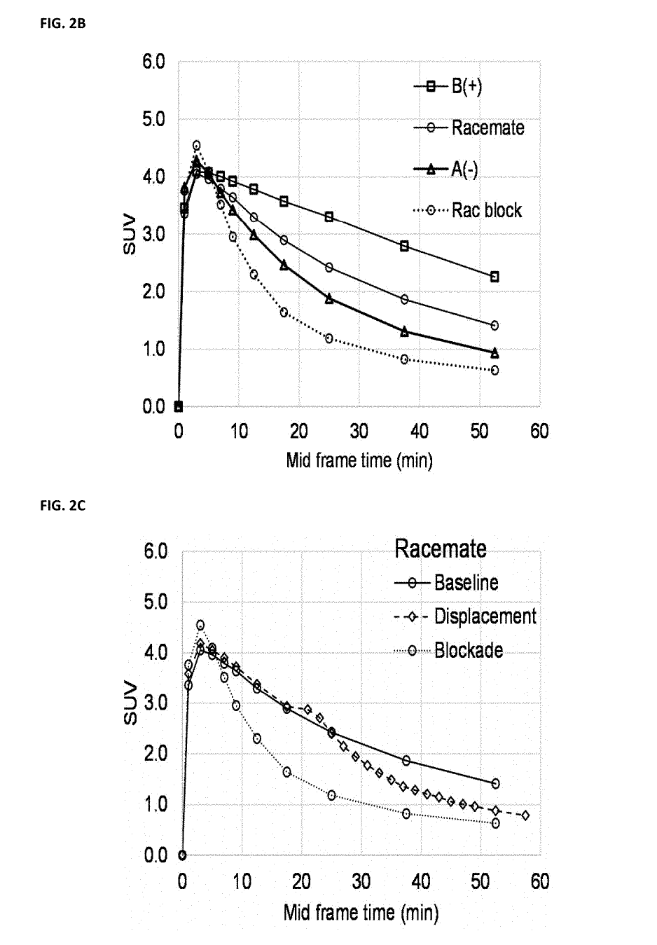

Wistar rats (331-353 g) were under isoflurane anaesthesia (2.5-5% in oxygen/air 1/1) for all procedures and temperature and respiration were controlled with warm air and by adjusting the isoflurane dosage. A dynamic PET scan was started with a calibrated VISTA eXplore/Super Argus PET/CT scanner (Sedecal, Madrid, Sprain; axial field of view 4.8 cm) and at time zero, 32-52 MBq (0.44-0.78 nmol/kg, 250-300 μL) [¹¹C]NB1 were injected via a tail vein. For blocking experiments by PET, injected doses of [¹¹C]NB1 were 20-67 MBq, the dose of eliprodil was 1 mg/kg. Images and decay-corrected time-activity curves (TAC) were generated in PMOD v3.6 (PMOD Technologies Ltd., Zurich, Switzerland) with predefined regions of interest (ROI) implemented in PMOD and as shown in FIGS. 2A, 2B, and 2C. [¹¹C]WMS1405 accumulates in NMDA-rich brain regions and is blocked and displaced, respectively, by the NMDA N2B-selective compound eliprodil. Stereoisomer B(+) accumulates more than the racemate, stereoisomer A(−) accumulates less than the racemate, both accumulate more than when the racemate is blocked by eliprodil.

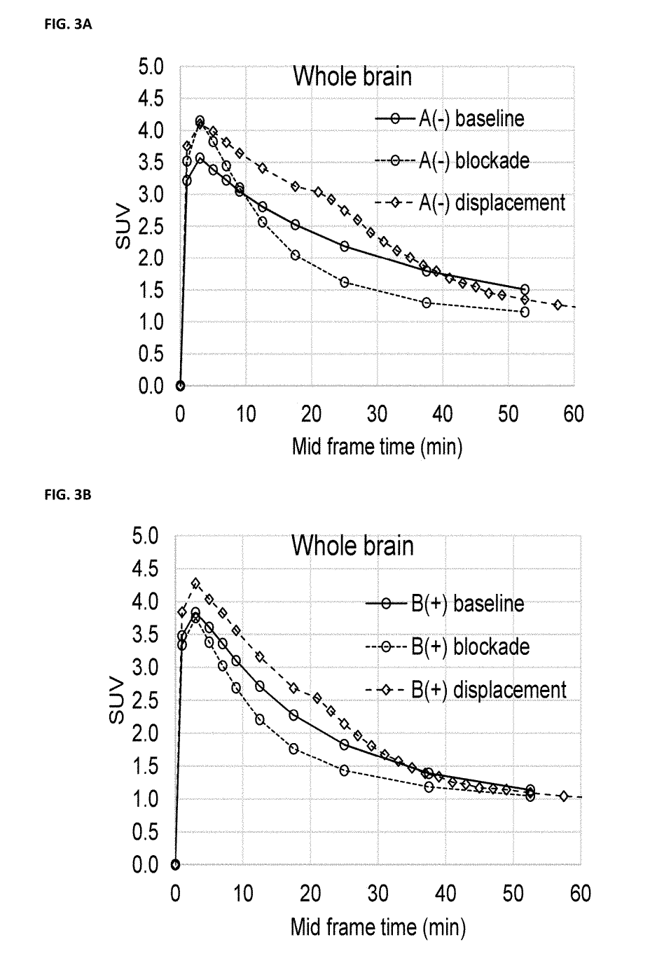

### Example 6—Time-Activity Curves of PET Scans of Rat Brain with Enantiomeric Pure [¹⁸F]WMS1410 Under Baseline and Blockade Conditions (FIGS. 3A and 3B)

Similar PET experimental procedure as mentioned above for [¹¹C]WMS1405 was applied for imaging [¹⁸F]WMS1410 with Wister rat. [¹⁸F]WMS1410 accumulates in NMDA-rich brain regions and is blocked by eliprodil. Both stereoisomers accumulate and are blocked and displaced, respectively, by eliprodil.

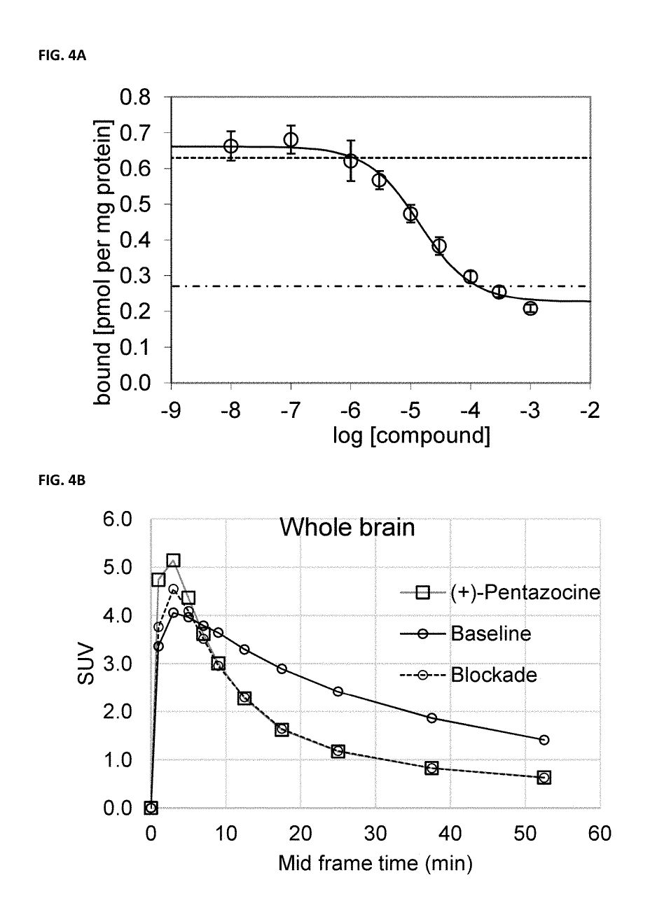

### Example 7—[¹¹C]WMS1405 Indirectly Images Modulation of NMDA Receptors by the Signal Receptor (FIGS. 4A and 4B)

In vitro binding study with [³H](+)pentazocine, a ligand for signal receptor. WMS1405 does not compete for the binding of (+)pentazocine to signal, excluding WMS1405 as a signal receptor ligand (Tewes B, Chem Med Chem. 2010, 5, 687-95).

In vivo, pentazocine and haloperidol, a ligand for several neuroreceptors including signal receptor, reduce [¹¹C]WMS1405 binding, albeit WMS1405 did not compete with pentazocine binding in vitro. This is strong evidence that PET with NR2B ligands such as WMS1405 or WMS1410 allows to indirectly image sigma receptor (in particular signal receptor) activity. It is known that signal receptor interacts with NMDA NR1A and modulates NMDA receptor function (Balasuriya D, J Neurosci. 2013, 33(46), 18219-24).

1. A method for the diagnosis of NMDA-receptor-associated diseases or disorders comprising the following steps:
(a) administering to a patient in need of such diagnosis a radioactively labelled compound in an amount effective for PET imaging of NMDA receptors,
(b) recording at least one PET scan, and
(c) diagnosing the patient as having an NMDA-receptor-associated disease or disorder from an abnormal NMDA receptor expression pattern on the PET scan,
wherein the radioactively labelled compound has formula (I) 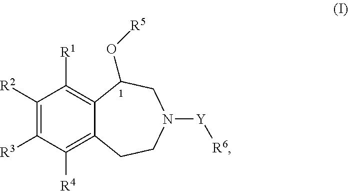
wherein
at least one atom of formula (I) is a radiolabeled atom, selected from the group of ¹¹C-, ¹⁸F-, ¹³N-, or ¹⁵O-atom, suitable for positron emission tomography detection (PET);
one of R¹, R², R³ and R⁴ is independently selected from the group consisting of hydrogen, fluorine, —(C₁-C₄)alkyl, fluorinated —(C₁-C₄)alkyl, —CH₂F,—CD₂F, FCH₂CH₂—, FCH₂CH₂CH₂—, —O(C₁-C₄)alkyl, fluorinated —O(C₁-C₄)alkyl, —OCH₂F,—OCD₂F, FCH₂CH₂O—, and FCH₂CH₂CH₂O—,
and the other of R¹ to R⁴ are hydrogen or fluorine;
R⁵ is selected from the group consisting of
hydrogen, —(C₁-C₆)alkyl, fluorinated —(C₁-C₆)alkyl, —CH₂F,—CD₂F, FCH₂CH₂—, and FCH₂CH₂CH₂—;
Y is selected from the group consisting of
(C₁-C₆)alkyl, preferably C₅-alkyl or C₄-alkyl,
(C₁-C₆)alkoxyalkyl, (C₁-C₆)polyethyleneglycoyl, (CH₂)₂—O—(CH₂)₂—R⁶, —(CH₂)₃—O—R⁶, —(CH₂)₄—O—R⁶,
(C₁-C₆)heteroalkyl, —(CH₂)₃—X—R⁶ and —(CH₂)₄—X—R⁶, wherein X is sulfur or SO₂, 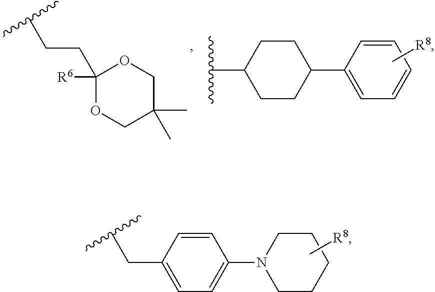
—(CH₂)₃—CO—R⁶, —(CH₂)₂—CO—N(CH₃)—CH₂—R⁶, and —CO—(CH₂)₃—R⁶, wherein R⁸ is one or more hydrogen or fluorine;
R⁶ is selected from the group consisting of
substituted or non-substituted (C₅-C₆)aryl, substituted or non-substituted (C₅-C₆)heteroaryl, substituted or non-substituted phenyl or pyridyl, and 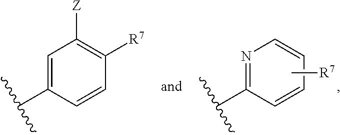
wherein Z is selected from the group consisting of hydrogen, fluorine and nitrile, wherein R⁷ is selected from the group consisting of hydrogen, fluorine, —(C₁-C₆)alkyl, fluorinated —(C₁-C₆)alkyl, —CH₂F,—CD₂F, FCH₂CH₂—, FCH₂CH₂—, and FCH₂CH₂CH₂—;
and pharmaceutically acceptable salts or solvates thereof.

2. The method of diagnosis according to claim 1, wherein the at least one radiolabeled atom is a ¹¹C-atom.

3. The method of diagnosis according to claim 1, wherein the NMDA-receptor-associated disease or disorder is selected from the group consisting of neurodegenerative diseases or disorders, Alzheimer's disease, depressive disorders, Parkinson's disease, traumatic brain injury, stroke, migraine, alcohol withdrawal and chronic and neuropathic pain.

4. The method of diagnosis according to claim 1, wherein at least one of R¹, R³ or R⁵ comprise a ¹¹C-atom.

5. The method of diagnosis according to claim 1, wherein one of R¹, R³ is —O[¹¹C]H₃ or R⁵ is —[¹C]H₃.

6. The method of diagnosis according to claim 1, wherein one of R¹, R², R³ and R⁴ are independently selected from the group consisting of —CH₃, —CH₂F,—CD₂F, FCH₂CH₂—, FCH₂CH₂CH₂—, —OCH₃, —OCH₂F,—OCD₂F, FCH₂CH₂O— and FCH₂CH₂CH₂O—, and the other of R¹ to R⁴ are hydrogen or fluorine.

7. The method of diagnosis according to claim 1, wherein R¹ is selected from the group consisting of —CH₃, —CH₂F,—CD₂F, FCH₂CH₂—, FCH₂CH₂CH₂—, —OCH₃, —OCH₂F,—OCD₂F, FCH₂CH₂O— and FCH₂CH₂CH₂O and R², R³ and R⁴ are hydrogen or fluorine

8. The method of diagnosis according to claim 1, wherein R³ is selected from the group consisting of —CH₃, —CH₂F,—CD₂F, FCH₂CH₂—, FCH₂CH₂CH₂—, —OCH₃, —OCH₂F,—OCD₂F, FCH₂CH₂O— and FCH₂CH₂CH₂O and R¹, R² and R⁴ are hydrogen or fluorine.

9. The method of diagnosis according to claim 1, wherein R⁵ is selected from the group consisting of hydrogen, —CH₂F,—CD₂F, FCH₂CH₂—, and FCH₂CH₂CH₂—.

10. The method of diagnosis according to claim 1, wherein Y is selected from the group consisting of —(CH₂)ᵢ—R⁶ and —(CH₂)ₑ—O—(CH₂)ₑ—R⁶, wherein i is an integer from 2 to 6, and e is an integer from 1 to 3.

11. The method of diagnosis according to claim 1, wherein R⁶ is selected from the group consisting of phenyl, 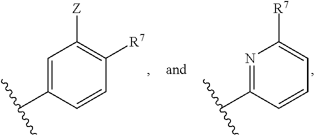
wherein Z is hydrogen or nitrile and wherein R⁷ is selected from the group consisting of fluorine, —CH₂F,—CD₂F, FCH₂CH₂—, FCH₂CH₂—, and FCH₂CH₂CH₂—.

12. The method of diagnosis according to claim 1, wherein the compound is R-configured at carbon 1.

13. The method of diagnosis according to claim 1, wherein the compound is S-configured at carbon 1.

14. The method of diagnosis according to claim 1, wherein
R¹ is selected from the group consisting of —OCH₃, —OCH₂F,—OCD₂F, FCH₂CH₂O— and FCH₂CH₂CH₂O and R², R³ and R⁴ are hydrogen,
R⁵ is hydrogen,
Y is —(CH₂)₄—R⁶, and
R⁶ is substituted or un-substituted phenyl.

15. The method of diagnosis according to claim 1, wherein
R³ is selected from the group consisting of —OCH₃, —OCH₂F,—OCD₂F, FCH₂CH₂O— and FCH₂CH₂CH₂O and R¹, R² and R⁴ are hydrogen or fluorine,
R⁵ is selected from the group consisting of hydrogen, —CH₂F,—CD₂F, FCH₂CH₂—, and FCH₂CH₂CH₂—,
Y is —(CH₂)₄—R⁶ or —(CH₂)₂—O—(CH₂)₂—R⁶, and
R⁶ is selected from the group consisting of phenyl, 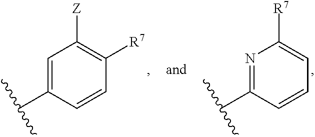
wherein Z is hydrogen or nitrile and wherein R⁷ is selected from the group consisting of fluorine, —CH₂F,—CD₂F, FCH₂CH₂—, FCH₂CH₂—, and FCH₂CH₂CH₂—.

16. The method of diagnosis according to claim 1, wherein
R³ is selected from the group consisting of fluorine, —CH₃, —CH₂F,—CD₂F, FCH₂CH₂— and FCH₂CH₂CH₂— and R¹, R² and R⁴ are hydrogen,
R⁵ is hydrogen,
Y is —(CH₂)₄—R⁶, and
R⁶ is substituted or un-substituted phenyl.

17. The method of diagnosis according to claim 1, wherein the compound is selected from the group consisting of 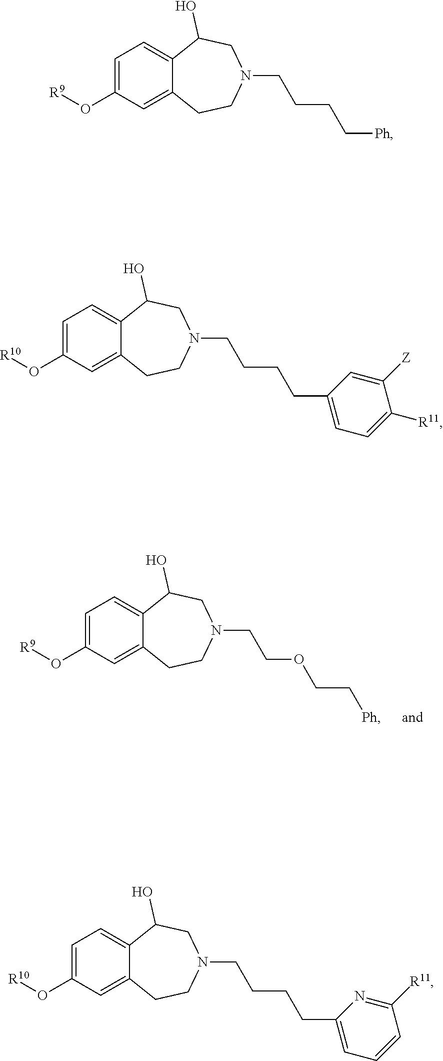
wherein
R⁹ is selected from the group consisting of CH₃, —CH₂F,—CD₂F, FCH₂CH₂—, FCH₂CH₂—, and FCH₂CH₂CH₂—;
R¹⁰ is selected from the group consisting of hydrogen, CH₃, —CH₂F,—CD₂F, FCH₂CH₂—, FCH₂CH₂—, and FCH₂CH₂CH₂—;
R¹¹ is selected from the group consisting of fluorine, —CH₂F,—CD₂F, FCH₂CH₂—, FCH₂CH₂—, and FCH₂CH₂CH₂—; and
Z is selected from the group consisting of hydrogen and nitrile.
